(19) United States
(12) Patent Application Publication
(10) Pub. No.: US 2018/0344329 A1
(43) Pub. Date: Dec. 6, 2018

(54) A MEDICAL DEVICE FOR SEPARATING TISSUES, A MEDICAL SYSTEM AND A METHOD THERETO

(71) Applicant: SURGIFY MEDICAL OY, Espoo (FI)

(72) Inventors: Seyedshahabaddin NAJAFI HAERI, ESPOO (FI); Visa SIPPOLA, HELSINKI (FI)

(73) Assignee: SURGIFY MEDICAL OY, Espoo (FI)

(21) Appl. No.: 15/780,481

(22) PCT Filed: Jun. 1, 2016
(86) PCT No.: PCT/FI2016/050392

§ 371 Date: May 31, 2018

Related U.S. Application Data
(60) Provisional application No. 62/261,537, filed on Dec. 1, 2015.

(51) Int. Cl. A61B 17/16; A61B 17/3203; A61B 17/17

(52) U.S. Cl. CPC A61B 17/1695; A61B 2017/320044; A61B 17/1739; A61B 17/3203

(57) ABSTRACT

Disclosed is a medical device for separating a first tissue from a second tissue. The medical device includes: a longitudinal stem part; a footplate part extending from one end of the longitudinal stem part; wherein at least one fluid channel is arranged to the medical device for providing a pressurized fluid for the separation of the first tissue from the second tissue, and wherein the medical device includes an interface for coupling the medical device to a surgical instrument used in a medical operation in which the first and the second tissue are separated. The invention relates also to a medical system including the medical device and a method using the same.

### TECHNICAL FIELD

The invention concerns in general the technical field of medical devices. More particularly, the invention concerns a medical device, a medical system and a method thereto for medical operation.

### BACKGROUND

Many times a treatment of human and animal bodies, for example with surgery, requires a separation of tissues of different types from each other. This is important because the aim in the treatment is to minimize any unnecessary impact to tissues that do not need any direct treatment e.g. in a form of surgery. For example, a craniotomy, as understood in this context, is a kind of operation in which a bone flap is temporarily removed from a skull in order to provide access to underlying tissues and the brain. Alternatively, in a craniectomy procedure, the bone flap is not immediately replaced. Craniotomy and craniectomy will be hereinafter referred to as craniotomy. Typically in the craniotomy one or more trephine holes are drilled to the skull with a trepanation instrument. The bone between the holes is then cut with a surgical instrument called craniotome having a rotary cutting tool at one end and a handle at the other end. After the holes have been connected, the bone flap is lifted to reveal the underlying tissues. As can be seen the aim in the craniotomy is to operate only the skull, not any other tissue, such as dura, below it. The same kind of situation is, for example, when a chest area or spine is to be operated and a surgical opening has to be created. There is sternum and directly below it are chest organs and their surrounding membranes which are not necessarily the target of the operation. Similarly, there is spine and below it the spinal cord and it's protecting membranes.

For the above mentioned situations there are available different kinds of medical devices by means of which the underlying tissues, such as dura, may be protected at least during the opening phase. An example of such a device used in craniotomy to protect dura is so called dura guard 100 as shown in FIG. 1. The dura guard 100 is adapted to be attached to the craniotome instrument 130 comprising a cutting tool, such as a rotary cutting tool 140. The dura guard 100 comprises a stem part 110 and a footplate part 120. The stem part is adapted to be coupled to the surgical instrument 130 from a first end. For example, it may comprise a locking mechanism by means of which the dura guard 100 may be fastened to the surgical instrument 130, as depicted in FIG. 1. The dura guard 100 continues from second end of the stem part 110 with a footplate 120. The footplate part 120 is adapted to extend underneath the cutting tool 140 for protecting the dura from the surgical instrument when the footplate 120 of the dura guard travels between the skull and the dura during the medical operation. Similar arrangements are used in other medical operations, as mentioned above.

However, the prior art medical devices, such as the dura guard 100, have some disadvantages. Namely, as the separation of the tissues, especially the hard tissue, such as bone, and the soft tissue, is based on a pure mechanical intervention with the medical device as described it may happen that the medical device, such as the dura guard, punctures the soft tissue, such as the dura, and the device enters to a space in the body that was not intended. This may cause further damages because the surgical instrument also cuts the soft tissue, like dura, in such a situation.

Further, the prior art solutions often requires that a plurality of holes are made to the hard tissue in order to bring in other instruments needed in the medical operation. For example, in skull operation a so called trephine hole is made for inserting the craniotome into the skull, but additional trephine holes are often necessary to insert other instruments under the skull to separate the dura from the skull prior to inserting the craniotome. Multiple trephine holes drilled during the craniotomy procedure consume time and effort and are a medical and cosmetic challenge to the patient.

Based on above it is clear that there is need to develop the prior art solutions in order to mitigate, at least partly, the disadvantages discussed above.

### SUMMARY

The following presents a simplified summary in order to provide basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

An objective of the invention is to present a solution for separating two tissues from each other. Another objective of the invention is that the solution according to the invention eases at least partly the medical operation in which the medical device is used.

The objectives of the invention are reached by a medical device, a medical system and a method as defined by the respective independent claims.

According to a first aspect, a medical device for separating a first tissue from a second tissue is provided, wherein the medical device comprises: a longitudinal stem part, a footplate part extending from one end of the longitudinal stem part, and wherein at least one fluid channel is arranged to the medical device for providing a pressurized fluid for the separation of the first tissue from the second tissue, and wherein the medical device comprises an interface for coupling the medical device to a surgical instrument used in a medical operation in which the first and the second tissue are separated.

The at least one fluid channel may be arranged in medical device with at least one of the following: at least one through hole arranged at least partly inside at least one of the following: the longitudinal stem part, the footplate part; at least one tube coupled to the medical device. The at least one tube may be arranged in at least one of the following: the at least one through hole arranged at least partly inside at least one of the following: the longitudinal stem part, the footplate part; at least partly on a surface of at least one of the following; the longitudinal stem part, the footplate part.

Further, at least one accommodation channel may be arranged at least partly on a surface of at least one of the following: the longitudinal stem part, the footplate part for accommodating the at least one tube in the at least one accommodation channel.

At least one output opening of the fluid channel for outputting the pressurized fluid may also be arranged at least in the footplate part. The at least one output opening of the at least one fluid channel may be implemented with the at least one through hole in the footplate part. Alternatively, the at least one output opening of the at least one fluid channel may be arranged in the footplate part by fixing at least an end of the tube to the footplate part.

Moreover, the at least one output opening may be arranged so that it is configured to output the fluid from a tip of the footplate part. The at least output opening formed by at least one of the following: an end of the at least one through hole, the end of the at least one tube in the tip of the footplate part may be arranged so that a central axis of the at least one of the following: an end of the through hole, the end of the at least one tube with respect to a longitudinal axis of the footplate part is acute.

At least one further output opening may be arranged as a through hole in at least one of the following: at least one side of the footplate part, a bottom of the footplate part, on top of the footplate part. The at least one further output opening residing on top of the footplate part may be connected to the output opening configured to output the fluid from the tip of the footplate part so that the further output opening is configured to operate as a valve for the output opening at the tip of the footplate part if the output opening is at least partly blocked.

The at least one output opening arranged as a through hole in the footplate part may have a tapered shape.

At least one further output opening may be arranged to the at least one tube by having at least one through hole on the surface of the at least one tube.

The at least one tube may be attached to the medical device with an adapter part arranged in the footplate part wherein the adapter part is configured to fasten at least the end of the at least one tube. The at least one tube may be adapted to the adapter part so that the fluid output from the at least one tube passes a shaped hole arranged in the adapter part for forming a pattern of the fluid when output.

The end of the at least one tube may be fastened to the medical device in one of the following way: with adhesive bonding if the tube is made of plastic; by laser welding or brazing if the tube is made of metal.

At least part of a surface of the medical device may be crimped for maintaining the at least one tube in its position.

At least a portion of the end of the at least one tube may be adjusted to form a nozzle generating a fluid stream in a specific form.

A nozzle insert may be arranged inside the end of the at least one tube to form a nozzle generating a fluid stream in a specific form.

A shape of the nozzle may be one of the following: circular, rectangular, elliptic.

The medical device may further comprise at least one outflow channel for the output fluid. The at least one outflow channel may be at least one of the following: a through hole arranged at least partly inside at least one of the following: the longitudinal stem part, the footplate part; a channel arranged at least partly on an outer surface of the medical device. The at least one outflow channel arranged at least on the outer surface of the medical device may be the accommodation channel.

The interface for coupling the medical device to the surgical instrument may be arranged in at least one of the following: the stem part, a matching part into which the stem part is adapted to.

The surgical instrument to which the medical device is to be coupled may be an instrument used for manipulating the first tissue.

The medical device may be a dura guard.

According to a second aspect, a medical system is provided, wherein the medical system comprising: a medical device as defined above, a pump device for providing fluid to a tube of the medical device, and a control device for controlling at least the provision of the fluid to the at least one tube of the medical device.

The medical system may further comprise a surgical instrument coupled with the medical device.

According to a third aspect, a method for separating a first tissue from a second tissue in a medical operation is provided, wherein the method comprises: outputting fluid between the first tissue and the second tissue with a medical device comprising: a longitudinal stem part, a footplate part extending from one end of the longitudinal stem part, wherein at least one fluid channel is arranged to the medical device for providing a pressurized fluid for the separation of the first tissue from the second tissue, and wherein the medical device comprises an interface for coupling the medical device to a surgical instrument used in a medical operation in which the first and the second tissue are separated.

Various exemplifying and non-limiting embodiments of the invention both as to constructions and to methods of operation, together with additional objects and advantages thereof, will be best understood from the following description of specific exemplifying and non-limiting embodiments when read in connection with the accompanying drawings.

The verbs “to comprise” and “to include” are used in this document as open limitations that neither exclude nor require the existence of unrecited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

The embodiments of the invention are illustrated by way of example, and not by way of limitation, in the figures of the accompanying drawings.

FIG. 1 illustrates a medical device according to a prior art solution applied with a surgical instrument.

FIGS. 2A-2C illustrate schematically some embodiments of a medical device according to the invention.

FIGS. 3A-3C illustrate schematically some further embodiment of a medical device according to the invention from different angles.

FIG. 4A-4C illustrate schematically still further embodiments of a medical device according to the invention.

FIGS. 5 illustrates schematically a still further embodiment of a medical device according to the invention.

FIGS. 6A-6C illustrate schematically some still further embodiments of a medical device according to the invention from one perspective.

FIGS. 7A-7C illustrate schematically the embodiments of a medical device according to the invention as shown in FIGS. 6A-6C from another perspective.

FIG. 8 illustrates schematically some aspects relating to a direction of a fluid stream output from a medical device according to the invention.

FIG. 9 illustrates schematically some further aspects relating to a medical device according to the invention.

FIG. 10 illustrates schematically an example of a medical device according to the invention in use.

FIG. 11 illustrates schematically an example of a medical system according to an embodiment of the invention.

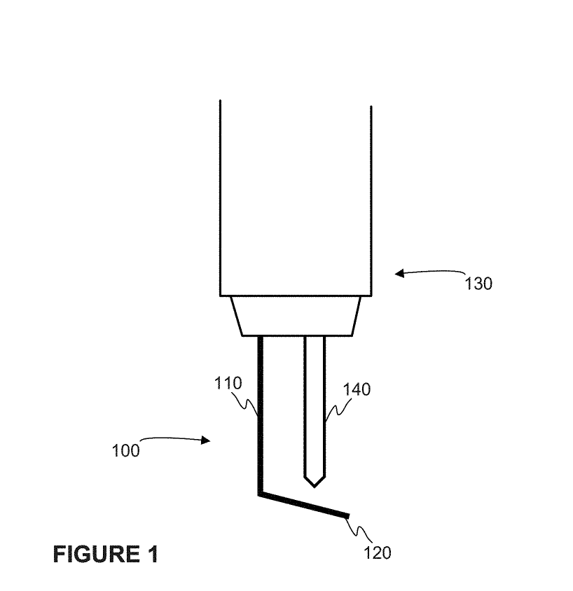

### DESCRIPTION OF THE EXEMPLIFYING EMBODIMENTS

The specific examples provided in the description given below should not be construed as limiting the scope and/or the applicability of the appended claims. Lists and groups of examples provided in the description given below are not exhaustive unless otherwise explicitly stated.

Some aspects of the present invention relates to a medical device for separating a first tissue from a second tissue during a medical operation, such as a craniectomy operation. Specifically, the medical device may especially be adapted to separate a soft tissue, such as a membrane, from a hard tissue, such as a bone. Typically, the membrane resides below the bone and the medical device may be needed in a situation when a medical intervention is made through the hard tissue and there is need to separate the soft tissue from the hard tissue for example in order to protect, at least partly, the soft tissue from any damage possible caused during the medical intervention. The medical device according to the example comprises two portions i.e. a stem part and the footplate part, wherein the footplate part extends from the stem part. Depending on an implementation of the medical device the stem part and the footplate part form a straight device or they may be formed at a predetermined angle with respect to each other. The stem part may be adapted to a matching part that may comprise necessary locking mechanism in order to attach the medical device either removable or permanently to some further device, such as to a surgical instrument. The locking mechanism may e.g. be threads arranged on an outer or inner surface of the matching part, which are adjusted so that they match with counter-threads arranged in the device, or similar, into which the medical device is to be fastened. As said the threads are one example of the locking mechanism and any other may be applied to. A specific matching part is not necessary in all implementation as the locking mechanism may e.g. be implemented directly to the stem part, e.g. by arranging threads into it. Thus, the medical device according to the present invention comprises an interface for coupling the medical device to a surgical instrument used in a medical operation in which the first and the second tissue are separated. The medical device is advantageously such an instrument which is used for manipulating the first tissue, such as an applicable cutting tool.

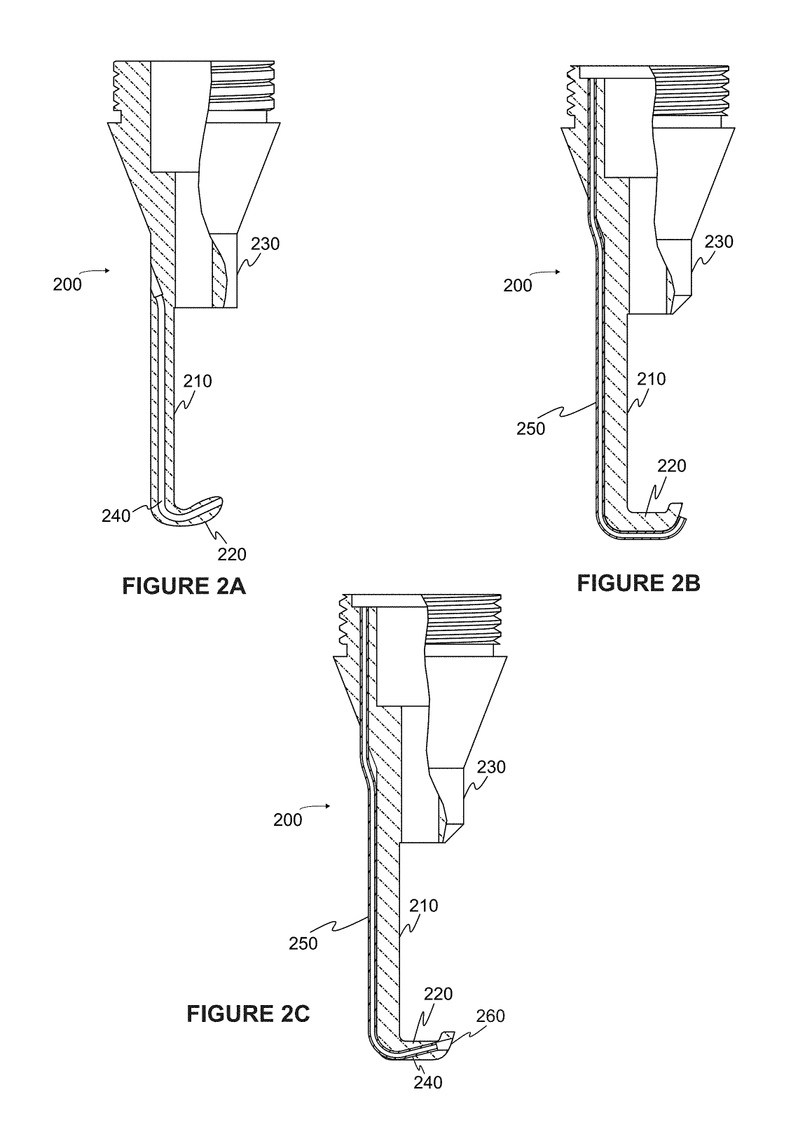

Further, the medical device according to the present invention comprises necessary arrangement for providing a pressurized fluid, preferably sterile fluid, for separating the mentioned tissues from each other, at least partly. The pressurized fluid may be provided through at least one fluid channel which is arranged to the medical device. FIGS. 2A-2C disclose embodiments of the medical device 200 according to the present invention wherein at least some alternatives for the implementation of the at least one fluid channel are shown. In the FIGS. 2A-2C the number of fluid channels is one, but the same inventive idea may be applied by providing a plurality (i.e. more than one) of fluid channels in the same manner. In the FIGS. 2A-2C the medical device 200 comprises at least the stem part 210 and the footplate part 220. The medical device may also comprise the matching part, as discussed above.

FIG. 2A discloses an embodiment of the medical device 200 in which at least one fluid channel is implemented with at least one through hole 240, which is arranged either inside at least one of the following: the footplate part 220, the longitudinal stem part 210. The through hole 240 does not necessarily travel along a full length of the stem part 210 and/or the footplate part 220.

FIG. 2B discloses another embodiment of the medical device 200 in which at least one fluid channel is implemented with at least one tube 250, wherein the at least one tube is arranged to travel along the medical device 200. The tube 250 may be fixed on an outer surface of the medical device 200 in some predetermined manner, e.g. with some mechanical fixing device, such as one or more strings or one or more clamps or laser welding, or by applying some chemical agent, such as adhesive, or any combination of these.

FIG. 2C discloses a still further embodiment of the medical device 200 in which at least one fluid channel is arranged by a combination of the embodiments according to FIG. 2A and 2B. In other words, a tube may be used to bring the fluid from some external device, such as a pump device, to some extent, wherein the tube is adjusted to a through hole 240 arranged in the footplate part 210. In some specific solution according to the embodiment of FIG. 2C at least one tube is arranged to a through hole in tube's full length. FIG. 2C also discloses an output opening 260 formed in the through hole 240.

The fluid channel, being either the through hole or the tube or the combination of these, is advantageously arranged to travel at least part of the length of the medical device 200. This applies to both when the fluid channel travels inside the medical device 200 or on the surface of it either partly or fully.

The medical device 200 according to the present invention may also comprise an adapter part at least in the stem part 210 especially when the fluid channel is implemented as a through hole 240. Namely, the adapter part in the stem part 210 provides an adapter for any external device, such as for a tube of a pump device, to couple with the medical device in order to provide the fluid in the fluid channel.

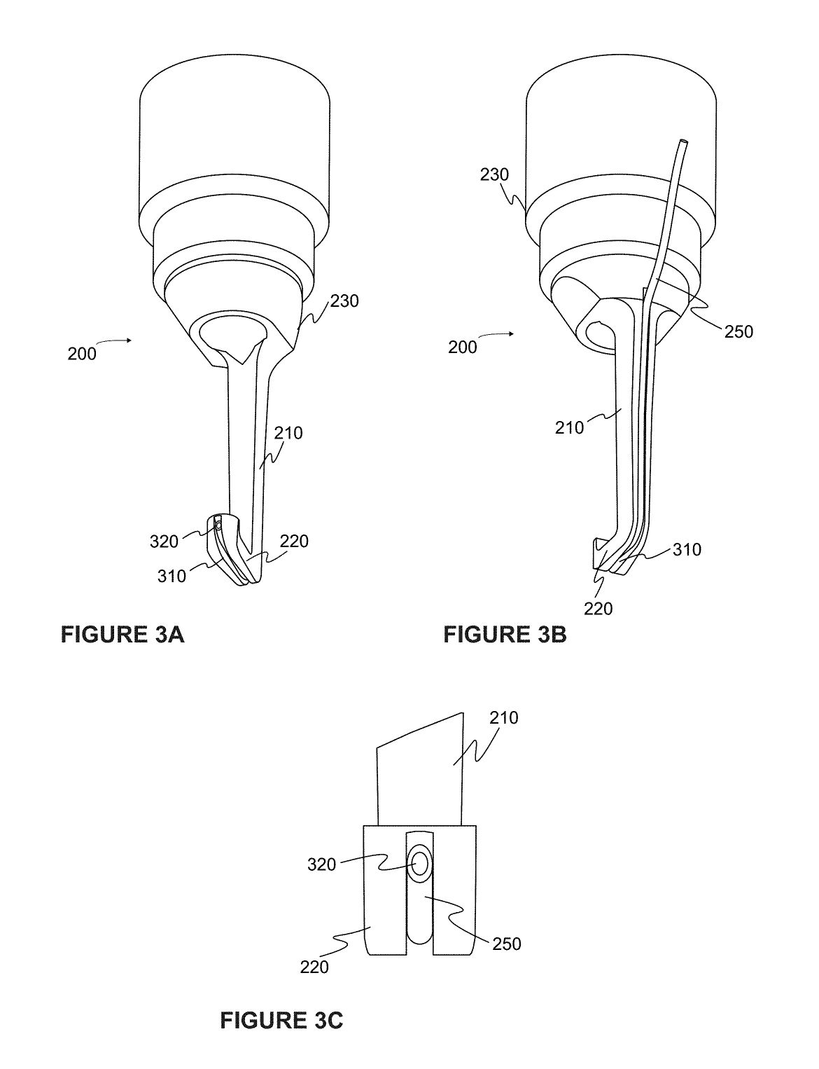

According to an embodiment of the invention the medical device 200 may comprise an accommodation channel for the at least one tube, when the at least one tube is arranged to travel at least partly on a surface of the medical device 200. An example of such an accommodation channel 310 is schematically illustrated in FIGS. 3A-3C from different angles. The accommodation channel 310 is advantageously shaped so that it may accommodate at least part of the at least one tube. Preferably, due to the operational environment, the accommodation channel is arranged to accommodate the at least one tube 250 in full, i.e. the tube travels in the channel 310 and does not extend out from the accommodation channel 310. The medical device 200 may comprise a plurality (i.e. more than one) accommodation channels 310 arranged in at least on portion of the medical device 200, or at least in a part of any portion. FIGS. 3A-3C also disclose an output opening 320 form by a mouth of the tube 250.

Next some further aspects on the accommodation of the at least one tube in the accommodation channel 310 is discussed. Namely, the at least one tube 250 may be accommodated in the accommodation channel 310 in multiple ways. Advantageously the end of the tube 250 is attached in one way or another to the end of the accommodation channel 310 with a fixing means so that a risk that the tube detaches during the use is minimized. For example the end of the tube 250 may be bonded adhesively to the fluid channel with an appropriate manner, especially if the tube is made of plastic. In case the tube is made of metal the attachment may e.g. be made by laser welding or brazing. Additionally, at least part of the inner walls of the accommodation channel, e.g. with the area wherein the end of the tube is positioned, or any other surface of the medical device, may be crimped, or grooved, so that the attachment of the tube, or at least a maintenance of the tube in its position, is improved. Alternatively or in addition to the above described methods to attach at least the end of the tube 250 in the accommodation channel 310 a separate adapter, such as a ring structure, may be arranged at the end of the accommodation channel 310 into which the end of the tube may be adapted to i.e. fastened. The adapter part may also have a structure that it comprises a shaped hole for forming a pattern of the fluid when output from the tube through the hole of the adapter part i.e. the adapter part becomes a part of the fluid channel. Even if the above described methods to attach the tube in the fluid channel are described in the context of the end of the tube, the similar attachments may also be arranged in any other part of the tube and the accommodation channel. For example, the tube may be attached along its full length to the accommodation channel into which it is accommodated. Moreover, in the context of the through hole the same attachment methods may be applied when the at least one tube 250 is adjusted in the through hole 240.

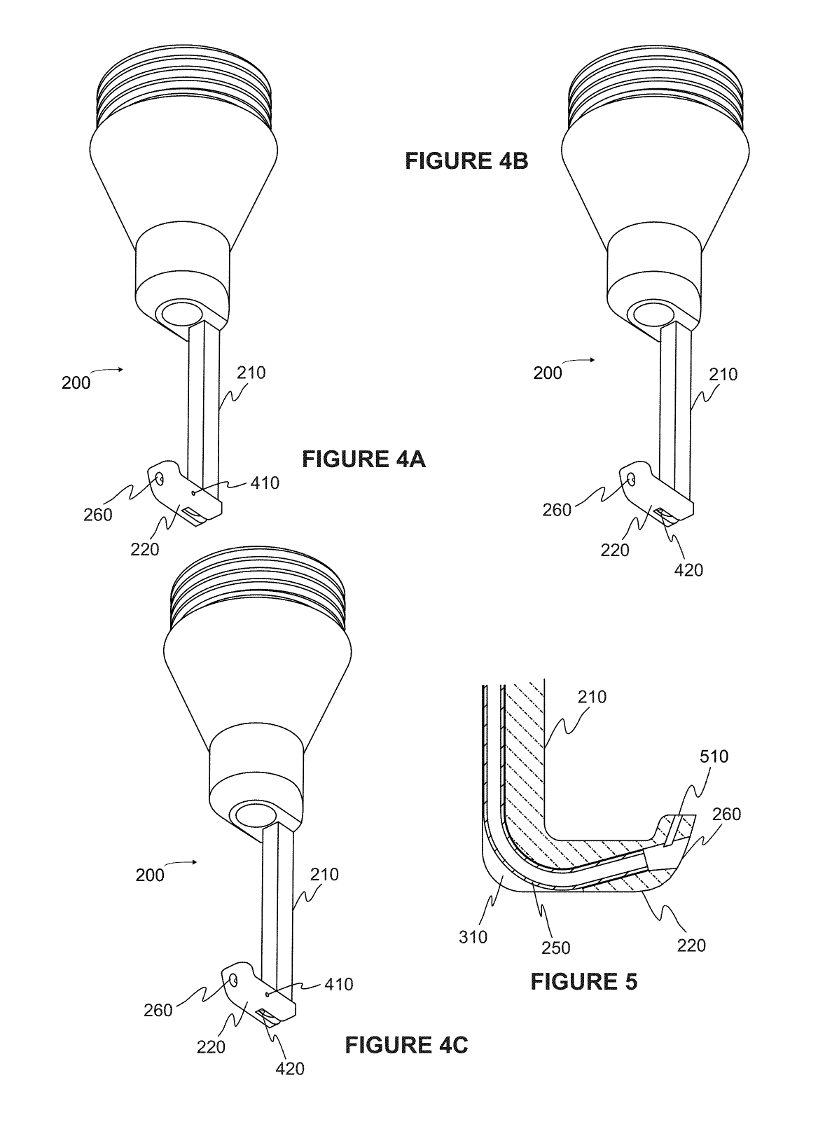

Next at least some aspects of one or more output openings 260, arranged in the medical device 200 are discussed. As said the medical device 200 according to the present invention may provide pressurized fluid in an environment where the medical device is used. As also mentioned an end of a through hole forms an output opening 260 in the footplate part 220 or the output opening may be formed by the tube 250, such as the mouth of the tube 250. According to some further embodiments of the medical device 200 according to the invention a plurality of output openings may be arranged for providing fluid to multiple directions. The further output openings may be arranged either to the footplate part 220 or to the tube 250 according to the implementation of the medical device 200. FIGS. 4A-4C disclose some further embodiments of the medical device 200 into which further output openings are arranged in addition to the one heading ahead from a tip of the footplate part 260. In the non-limiting examples of FIGS. 4A-4C the further output openings are disclosed in such an embodiment of the medical device 200 in which an accommodation channel 310 is arranged for a tube 250, which tube 250 is further taken to a through hole 240 in the footplate part 220.

In FIG. 4A it is shown an embodiment of the invention in which the medical device 200 comprises a further output opening 410 at least one side of the footplate part 220 of the medical device 200. The output opening 410 may advantageously be connected to the same fluid channel as the output opening 260 facing out from the tip of the footplate part 220.

FIG. 4B, in turn, illustrates schematically another embodiment of the invention in which a further output opening 420 is arranged on the bottom side of the footplate part 220 so that it provides the fluid downwards when the fluid is input to the medical device with a pump device or similar. As a matter of fact the non-limiting example of the output opening 420 according to the implementation of FIG. 4B is arranged to the tube 250 as a through hole on the surface that faces the open side of the accommodation channel, and as a result the fluid stream is discharged out from the tube 250 in the position of the through hole implemented in the tube 250. Alternatively or in addition the footplate part 220 may comprise a specific output opening, i.e. other than the output opening 420 arranged in the tube, in the bottom side.

FIG. 4C illustrates schematically a still further embodiment of the invention into which all the previously described output openings 260, 410, 420 are implemented in. Also in the example of the invention as depicted in FIG. 4C the output openings 410, 420 may advantageously, but not necessarily, be connected to the same fluid channel as the output opening 260.

The non-limiting embodiments of the invention shown in 4A-4C in which there are arranged multiple output openings in the medical device 200 are examples and it is possible to arrange one or more further openings to further positions if seen necessary. This kind of need may e.g. originate from a medical operation in which the medical device according to the present invention is to be needed. Advantageously, mutual sizes of the openings 260, 410, 420 are such that advantageously the main opening 260 has a bigger diameter than the side 410 or bottom 420 openings. Furthermore, the side opening may have a larger diameter than the bottom opening. By sizing the openings mutually it is possible to adjust and control at least partly the operation of the medical device i.e. how much fluid is provided in the space per each opening. In case the footplate part 220 of the medical device 200 comprises at least one of the openings in side or in bottom a corresponding holes may be arranged in the tube 250 if the tube forms the fluid channel in the corresponding position to the output opening in question.

FIG. 5 illustrates a specific embodiment of the present invention in which a further output opening 510 is arranged on top of the footplate part 220, and wherein the further output opening 510 is connected to the output opening 260 i.e. both output openings 260, 510 output fluid from the same fluid channel. The advantage of this kind of arrangement is that the further output opening 510 may operate as a safety valve which acts if the output opening being a main output opening in this regard gets blocked e.g. by a soft tissue. In this kind of situation the further output opening 510 provides a path for the fluid to escape from the fluid channel and thus mitigates a risk that the medical device, and the medical system as a whole, gets damaged. Advantageously, the further output opening 510 may be implemented in the hook portion of the footplate part 220, or on the top surface of the footplate part 220, heading the fluid stream upwards therefrom. The output opening 510 is such a location does not get blocked that easily. The operation of the output opening, i.e. the amount of fluid to be output from the further output opening 510 in a normal operation, may be adjusted by dimensioning the mutual diameters of the output openings 260, 510 optimally. The further output opening 510 may be implemented in any embodiment of the medical device wherein there is need to provide a valve for any output opening. However, the further output opening 510 is not necessary in every implementation of the medical device according to the present invention. The further output opening 510 may be implemented by drilling or by laser cutting it thereto, for example.

The shape of the output openings, especially of those which are implemented in the footplate part 220 at the end of the through hole 240 may vary. Advantageously the output opening of the through hole in the footplate part 220 may be tapered, as illustrated as a cone-shaped structure e.g. in FIG. 5. Such a shape increases room for the fluid to escape because the soft tissue in front of the output opening may block at least part of the output opening. Hence, having a larger output opening 260 may be advantageous.

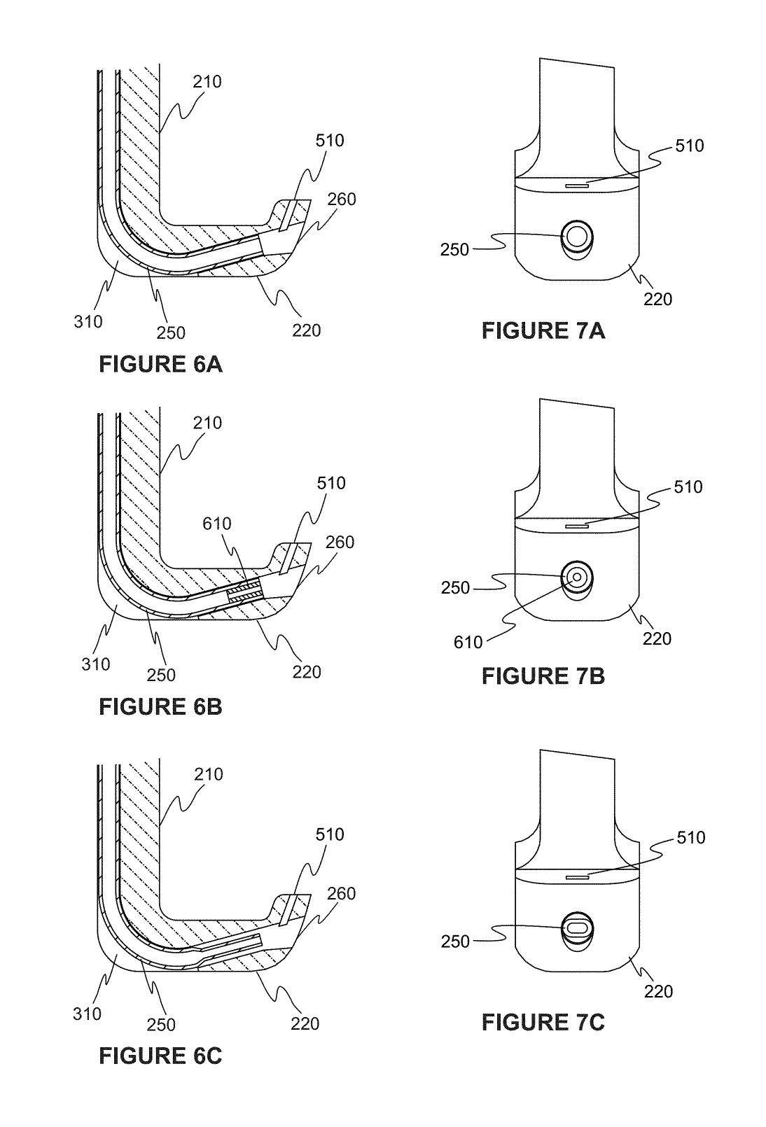

In the FIGS. 6A-6C and 7A-7C it is schematically illustrated some aspects relating to the output opening formed by the tube 250, and specifically the mouth of the tube 250. The examples are described in an embodiment of the medical device 200 which comprises the accommodation channel 310 and a through hole 240 in the footplate part 220 into which the tube is adjusted. The output of the fluid from the tube 250, or from the output opening formed by the mouth of the tube, may be performed with different stream patterns. The varied stream patterns may be implemented by shaping at least a portion of the end of the tube 250 in a manner that generates the stream pattern. FIGS. 6A-6C illustrate schematically some possible shapes at the end of the tube 250 that generate an applicable fluid stream pattern. The section shape of the tube 250 in FIG. 6A is essentially circular that generates a solid stream pattern from the output opening of the medical device 200. In the implementation as depicted in FIG. 6B a nozzle insert 610, such as a cylindrical part with an opening, is installed at the tubing end. The nozzle insert 610 may be shaped according to any need, for example being rectangular or elliptic generating a flat fan shaped stream. The nozzle insert may be mounted e.g. by laser welding or press fitting among any other applicable way. FIG. 6C, in turn, illustrates a solution in which a shape of the tubing end is modified, such as crimped, in order to shape the section so that a desired fluid stream pattern may be achieved. For example, the tubing end may be shaped oval or rectangular in order to generate a flat fan shaped stream. In other words, the shape of the end of the tube 250 operates as a nozzle for shaping the output fluid stream. The above mentioned patterns are only examples, and further patterns may be implemented by shaping the end of the tube 250 or implementing a nozzle insert accordingly. Non-limiting examples of the generated stream patterns are hollow cone, full cone and atomized stream. Moreover, the nozzle insert 610 may also be included in a tube that is already formed in a predetermined shape.

FIGS. 7A-7C illustrate schematically the shapes of the tubing end of FIGS. 6A-6C, correspondingly from front of the footplate part 220. The FIGS. 7A-7C provide a better view on shape and deepens the information of FIGS. 6A-6C. As may be seen in the embodiments in which the tube 250 is arranged to operate as the fluid channel and it is adjusted to a through hole in the footplate part the tube 250 advantageously ends at an applicable distance from the output opening 260. This is because energy of the fluid stream near the exit in the tubing end is very high and it is good to have some space, or distance, between the tubing end and the soft tissue for avoiding, or at least reducing a risk to, damage the soft tissue, or any other tissue in the environment.

As already mentioned a separate adapter part into which the tube 250 may be adjusted from its mouth may comprise the corresponding nozzle shapes implemented in the similar way, as described above.

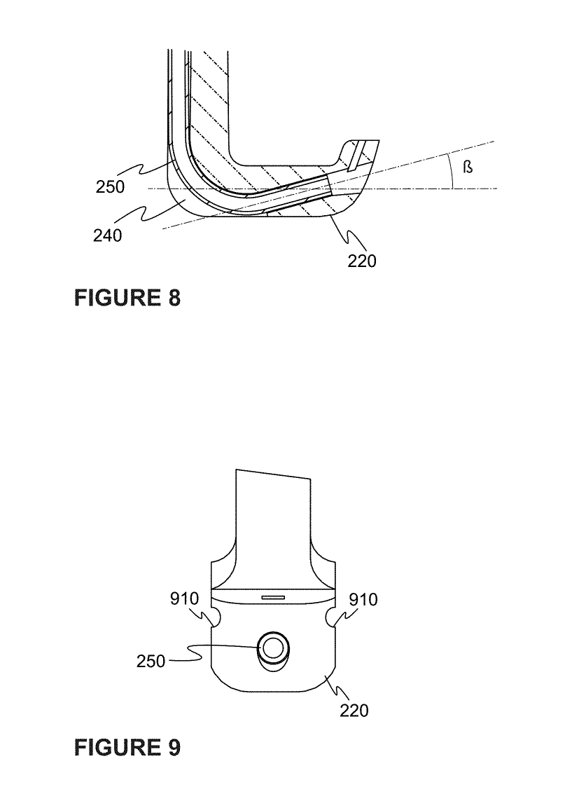

Next at least some aspects regarding an advantageous direction, in at least some application area, of a fluid stream output e.g. from the output opening implemented in the tip of the footplate part 220 are discussed by referring to FIG. 8, which depicts an embodiment in which a tube 250 performs an operation of the fluid channel and the end of the tube is adjusted in a through hole implemented in the footplate part 220. According to the embodiment of FIG. 8 the end of the tube 250 is adjusted in such a manner to its position that the fluid stream output from the tube 250 from the main opening, i.e. from the end of the tube 250, is directed upwards with respect to a surface of the tissue facing the bottom of the footplate part 220. In other words the angle β of a central axis of the end of the tube 250 with respect to a longitudinal axis of the footplate part 220 extending out from the tip of the footplate part 220 is preferably acute. The angle β is shown in FIG. 8 for clarifying purposes. The upward direction of the fluid stream is thus achieved by arranging at least the end of the tube 250 to head in the upward direction. In the implementation of the invention in which the tube 250 is inserted to a through hole arranged in the footplate part 220 which through hole ends at the output opening 260, the output opening 260 may be formed accordingly i.e. it does not prevent the fluid stream to output to the desired direction defined by the positioning of the end of the tube 250 in the footplate part 220. By arranging the direction of the fluid stream upwards as described it is possible to reduce, at least partly, a risk that the fluid stream hits the underlying tissue when the medical device is tilted in use. This prevents, at least partly, damaging the soft tissue, such as the dura and/or brain tissue below the dura, with the fluid stream output from the tube 250 and/or any output opening especially when the dura is already damaged and the footplate part 220 is in a subdural space. An advantageous angle of the fluid stream, and hence the end of the tube 250, with respect to the longitudinal axis of the footplate part 220 may e.g. be 20-60 degrees in order to output the fluid to the space in the mentioned angle. The same consideration as described above in the context of the embodiment as shown in FIG. 8 may be applied in embodiments where no tube 250 is used, e.g. the embodiment as shown in FIG. 2A. In other words, the direction of the fluid channel, especially at its end, is advantageously implemented so that it provides the fluid stream to upward direction with respect to a longitudinal axis of the footplate part 220.

Some aspects of the present invention relate to a solution for arranging an outflow of the fluid input to the environment, such as in an intracranial space in a skull operation. For example, if the medical device is used in craniotomy, i.e. as a dura guard, the fluid brought in to a subdural space shall advantageously be outflowed from the volume in order to mitigate a risk that intracranial pressure would rise dangerously due to input fluid accumulation. Thus, the medical device 200 according to a further embodiment of the invention may comprise at least one outflow channel for discharging at least part of the fluid output in the environment where the medical device is used for. The at least one outflow channel may be at least one of the following: an outflow through hole arranged at least partly inside at least one of the following: the longitudinal stem part 210, the footplate part 220; a channel arranged at least partly on an outer surface of the medical device 200. In other words, the medical device may comprise one or more fluid channels by means of which the fluid is provided by the medical device and one or more outflow channels in order to discharge the fluid at least partly. The outflow channel may be at least through implemented inside at least a portion of the medical device, which enables the discharging of fluid from the operational area of the device. Alternatively or in addition, one or more channels are arranged on an outer surface of the medical device 200 for providing a path for the fluid to “escape” from the volume into which it is output, as referred with 910 in FIG. 9. Such channels may e.g. be grooves with applicable dimensions, or if the medical device comprises the accommodation channel 310 for the at least one tube 250 it may be dimensioned so that it may simultaneously provide an outflow channel for the fluid (see e.g. FIG. 3C). The outflow of the fluid may e.g. be a result of pressure difference between the ends of the outflow channel. In some embodiment of the present invention suction may be arranged from the distal end of the outflow channel in order to cause the fluid to escape from the volume into which it is output through the one or more outflow channels. As said, one or more outflow channels may be implemented in any embodiment of the present invention in the manner as described.

Naturally, in order to manage the fluid accumulation the speed of the fluid stream, and thus the amount of fluid to be provided, may be controlled so that it optimizes a fluid accumulation in an intracranial space. Preferably it is minimized, but most importantly the maximum inflow does not advantageously exceed the outflow capacity of the one or more outflow channels.

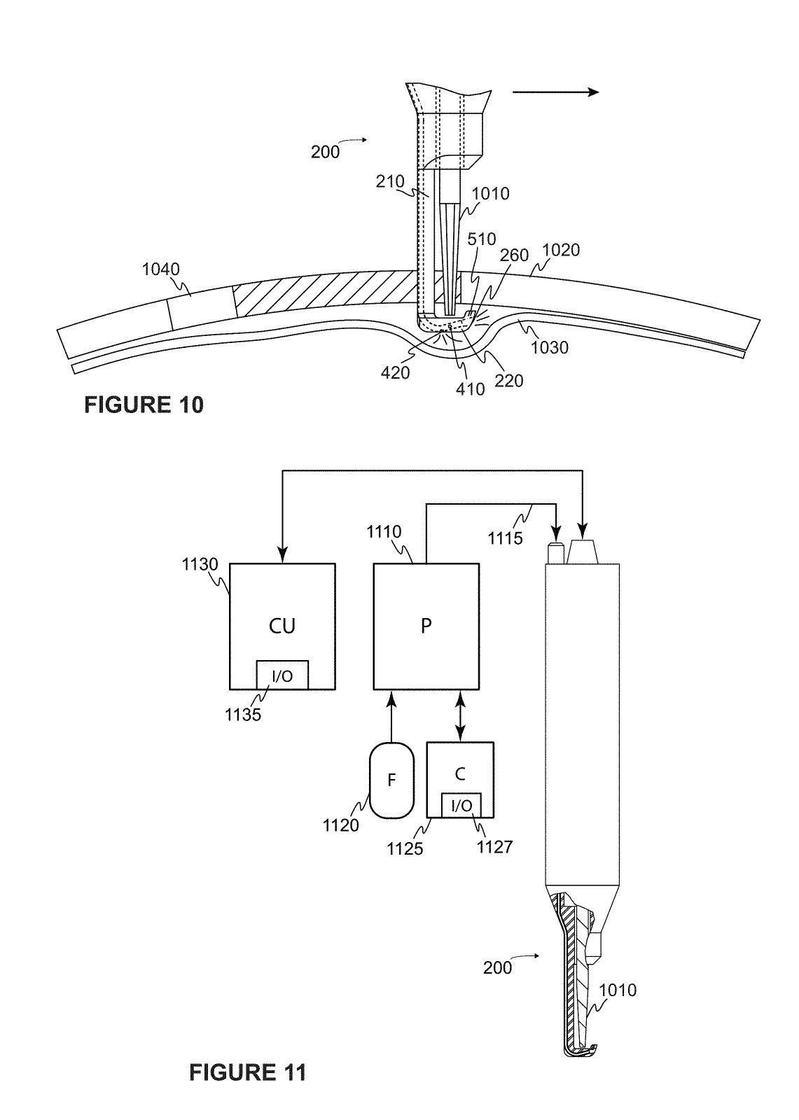

FIG. 10 illustrates schematically an example of the medical device 200 according to an embodiment of the present invention used in a context of craniotomy operation. The medical device 200 in the example may be considered to operate as a dura guard. The medical device comprising a stem part 210 and the footplate part 220 wherein the footplate part 220 comprises a plurality of output openings 260, 410, 420, 510 for outputting fluid input to the medical device. The medical device 200 comprises an interface for coupling the medical device to a surgical instrument, such as to a craniotome having a rotary cutting tool as a surgical instrument 1010. FIG. 10 also illustrates the skull 1020, i.e. the hard tissue, and the dura 1030, i.e. the soft tissue. The arrow in FIG. 10 refers to a direction into which the medical device with the surgical instrument is moved to.

The craniotomy operation of FIG. 10 starts by preparing a trephine hole 1040 to the skull with a specific tool in order to bring the surgical instrument 1010 with the medical device into the operational position. This means that the bottom of the footplate part 220 of the medical device is towards the dura 1030 and the footplate part 220, at least partly, resides between the skull 1020 and the dura 1030. By providing fluid from the output openings 260, 410, 420, 510 it is possible to enhance the separation of the dura 1030 from the skull and in that manner to reduce the risk to damage the dura with a mechanical interaction. In other words, the medical device 200 with the input fluid forces the dura 1030 to separate at least partly from the skull 1020 and in that manner protect the dura, at least partly, from the surgical instrument 1010 and the mechanical interaction with the footplate part 220. In response to the provision of the fluid the cutting operation with the surgical instrument 1010, such as rotary cutting tool, may be initiated and performed, as schematically illustrated in FIG. 8.

FIG. 11 illustrates schematically a medical system according to an embodiment of the invention. The medical system comprises the medical device as described above. Fluid may be input to the medical device 200 from a pump device 1110 through a delivery tubing 1115 that is advantageously flexible and coupled to the fluid channel or to the tube 250. Between the delivery tubing 1115 and the tube 250 may be an adapter. According to a further embodiment of the invention the delivery tubing 1115 and the tube 250 may be the same element. The pump device 1110 comprises necessary parts and elements that enable the provision of the fluid into the medical device 200. A fluid may be provided to the pump device 1110 from a fluid storage 1120. The operation of the pump device 810 may be controlled with a control device 1125. The control device 1125 comprises necessary input and output devices 1127 for enabling the operator, such as a surgeon, to operate the pump device 1110. For example, the operator may control the input of the fluid to the medical device 200 with a foot pedal or a control handle as I/O devices. The control handle may be implemented in the context of the medical device 200, or the surgical instrument, enabling the operator to control the input of the fluid when using the medical device 200 and/or the surgical instrument. Controllable parameter by the operator may e.g. be a fluid pressure having effect to the flow of the fluid. Moreover, the solution may also comprise a control unit 1130 for controlling the surgical instrument, which controls e.g. the rotary cutting tool speed. Similarly, the control unit 1130 may comprise input/output devices 1135 for providing the control signals by the operator as well as receiving one or more parameters representing an operational state of the surgical instrument. For example, the control devices 1125 and 1130 may be implemented with a processor and a memory storing computer program code and necessary parameters. By executing the computer program code with the processor control signals may be generated in order to achieve the desired operation for inputting the fluid. Generally speaking in this context the pump device 1110 shall advantageously be considered to cover both the fluid storage 1120 and the control unit 1125, which control device 1125 may also comprises necessary devices and function to operate the surgical instrument. Hence, the medical system comprises advantageously a control device 1125, a pump device 1110 and the medical device 200 coupled together.

The fluid to be input is advantageously liquid, but at least in some context an applicable gas may also be input. An example of the fluid is isotonic 0.9 sterilized saline, which may additionally contain other solvents, such as coagulation factors or abrasive particles.

In some further example the medical system 200 may comprise a suction device, such as a pump device, for enabling a suction of the fluid at least partly from the space in which the medical device 200 is operated through an outflow channel, for example. The fluid to be suctioned may be the same that is input by the medical device 200 or any other existing in the space. Naturally, the system shall comprise a suction tubing couplet to the suction device for performing the suction.

In the examples referred above the accommodation channel is at least partly open traveling at least partly along at least the stem part and/or footplate part. It is also possible to arrange bridge sections over the accommodation channel in order to enhance the positioning of the tube in the accommodation channel as well as use any attachment means for accommodation of the tube in the accommodation channel. The bridge sections preferably travel over the accommodating channel so that it may support the tube from the open side of channel. The number of bridge sections is not limited. Moreover, the medical device may also comprise output openings in other sections of the device, such as in stem part or even in the matching part e.g. if there is need to provide the fluid from that direction in the medical operation in question. Alternatively or in addition, a number of the output openings in different sections, or locations, of the medical device 200 may vary. For example, multiple output openings may be arranged at the tip of the footplate part.

The invention is described herein mainly with such an implementation in which only one fluid channel is disclosed. This does not limit the invention and it is possible to implement the invention so that there are arranged multiple fluid channels in the medical device.

The medical device 200 according to the present invention provides an advantage that by inputting fluid during the operation it is possible to enhance the operation of the medical device. In other words, by inputting the fluid with appropriate pressure and flow it is possible to enhance the separation of the tissues. The structures of the medical device as illustrated in the enclosed figures are non-limiting examples and the structure may vary. For example, the matching part 230 is not necessary in all implementations. Moreover, the first end of the stem part 210 may be equipped with an interface, such as a locking mechanism, and be directly mountable to any other device if needed. Hence, the medical device according to at least some example may comprise an interface for mounting it either removably or permanently to a least one further device. Moreover, the medical device 200 according to some examples of the present invention may also be used without mounting it to any other device.

The effect of the present invention is at least partly based on a mechanical force of the fluid stream when input in the space with the controllable pressure. The input fluid, and specifically the fluid stream output from the bottom opening of the footplate, reduces at least partly frictional forces between the medical device 200 and the soft tissue, such as a dura. Another advantage of at least some embodiments of the present invention is that the at least partly open fluid channel enables cleaning and sterilization of the medical device. Moreover, the replacement of a tube 250 may also be made in such implementations, especially if the attachment is such that the tube 250 is easy to detach from the stem part 210 and/or from the footplate part 220.

For sake of clarity in the following some non-limiting dimensions are given for at least some embodiment of the medical device according to the present invention. For example, the stem part 210 may be 12-21 mm long and depends at least on the patient age and operation location. The footplate part 220 may e.g. be 6 mm in length. The fluid channel 350 may be 0.8 mm wide into which the tube is to be adjusted. The tube 360 may have inner diameter of 0.1-0.4 mm and outer diameter of 0.3 mm to the diameter equal to the channel size, for example. The fluid is advantageously output with a pressure of 30-200 bar. The dimensions given above are non-limiting examples in order to give the reader some idea of possible dimensions of the medical device. The material of the medical device may be metal or plastic, at least partly or in full.

The specific examples provided in the description given above should not be construed as limiting the applicability and/or the interpretation of the appended claims. Lists and groups of examples provided in the description given above are not exhaustive unless otherwise explicitly stated.

1-29. (canceled)

30. A medical device for separating a first tissue from a second tissue, comprising:
a longitudinal stem part,
a footplate part extending from one end of the longitudinal stem part,
wherein at least one fluid channel is arranged to the medical device for providing a pressurized fluid for the separation of the first tissue from the second tissue, and
wherein the medical device comprises an interface for coupling the medical device to a surgical instrument used in a medical operation in which the first and the second tissue are separated.

31. The medical device of claim 30, wherein the at least one fluid channel is arranged in medical device with at least one of the following: at least one through hole arranged at least partly inside at least one of the following: the longitudinal stem part, the footplate part; at least one tube coupled to the medical device.

32. The medical device of claim 31, wherein the at least one tube is arranged in at least one of the following: the at least one through hole arranged at least partly inside at least one of the following: the longitudinal stem part, the footplate part; at least partly on a surface of at least one of the following; the longitudinal stem part, the footplate part.

33. The medical device of claim 31, wherein at least one accommodation channel is arranged at least partly on a surface of at least one of the following: the longitudinal stem part, the footplate part for accommodating the at least one tube in the at least one accommodation channel.

34. The medical device of claim 30, wherein at least one output opening of the fluid channel for outputting the pressurized fluid is arranged at least in the footplate part.

35. The medical device of claim 34, wherein the at least one output opening of the at least one fluid channel is implemented with the at least one through hole in the footplate part.

36. The medical device of claim 34, wherein the at least one output opening of the at least one fluid channel is arranged in the footplate part by fixing at least an end of the tube to the footplate part.

37. The medical device of claim 34, wherein the at least one output opening is arranged so that it is configured to output the fluid from a tip of the footplate part.

38. The medical device of claim 37, wherein the at least output opening formed by at least one of the following: an end of the at least one through hole, the end of the at least one tube in the tip of the footplate part is arranged so that a central axis of the at least one of the following: an end of the through hole, the end of the at least one tube with respect to a longitudinal axis of the footplate part is acute.

39. The medical device of claim 34, wherein at least one further output opening is arranged as a through hole in at least one of the following: at least one side of the footplate part, a bottom of the footplate part, on top of the footplate part.

40. The medical device of claim 39, wherein the at least one further output opening residing on top of the footplate part is connected to the output opening configured to output the fluid from the tip of the footplate part so that the further output opening is configured to operate as a valve for the output opening at the tip of the footplate part if the output opening is at least partly blocked.

41. The medical device of claim 34, wherein at least one further output opening is arranged to the at least one tube by having at least one through hole on the surface of the at least one tube.

42. The medical device of claim 31, wherein the at least one tube is attached to the medical device with an adapter part arranged in the footplate part wherein the adapter part is configured to fasten at least the end of the at least one tube.

43. The medical device of claim 42, wherein the at least one tube is adapted to the adapter part so that the fluid output from the at least one tube passes a shaped hole arranged in the adapter part for forming a pattern of the fluid when output.

44. The medical device of claim 36, wherein the end of the at least one tube is fastened to the medical device in one of the following way:
with adhesive bonding if the tube is made of plastic,
by laser welding or brazing if the tube is made of metal.

45. The medical device of claim 30, wherein at least part of a surface of the medical device is crimped for maintaining the at least one tube in its position.

46. The medical device of claim 30, wherein at least a portion of the end of the at least one tube is adjusted to form a nozzle generating a fluid stream in a specific form.

47. The medical device of claim 30, wherein a nozzle insert is arranged inside the end of the at least one tube to form a nozzle generating a fluid stream in a specific form.

48. The medical device of claim 30, wherein the medical device further comprises at least one outflow channel for the output fluid.

49. The medical device of claim 48, wherein the at least one outflow channel is at least one of the following: a through hole arranged at least partly inside at least one of the following: the longitudinal stem part, the footplate part; a channel arranged at least partly on an outer surface of the medical device.

50. The medical device of claim 49, wherein the at least one outflow channel arranged at least on the outer surface of the medical device is the accommodation channel.

51. The medical device of claim 30, wherein the interface for coupling the medical device to the surgical instrument is arranged in at least one of the following: the stem part, a matching part into which the stem part is adapted to.

52. The medical device of claim 30, wherein the surgical instrument to which the medical device is to be coupled is an instrument used for manipulating the first tissue.

53. The medical device of claim 30, wherein the medical device is a dura guard.

54. A medical system, the medical system comprising:
a medical device as defined in claim 30,
a pump device for providing fluid to a tube of the medical device, and
a control device for controlling at least the provision of the fluid to the at least one tube of the medical device.

55. The medical system of claim 54, the medical system further comprising a surgical instrument coupled with the medical device.
